(19) United States
(12) Patent Application Publication
(10) Pub. No.: US 2016/0271062 A1
(43) Pub. Date: Sep. 22, 2016

(54) MICELLE COMPOSITIONS AND PROCESS FOR THE PREPARATION THEREOF

(71) Applicant: DSM IP ASSETS B.V., Heerlen (NL)

(72) Inventors: Jerome George Jozeph Louis LEBOUILLE, Munsterbilzen (BE); Tessa KOCKELKOREN, Beek (NL); Leopold Franciscus Wijnandus VLEUGELS, Beek (NL); Remco TUINIER, Sittard (NL)

(21) Appl. No.: 15/159,736

(22) Filed: May 19, 2016

Related U.S. Application Data
(63) Continuation of application No. 13/634,445, filed on Nov. 26, 2012, now abandoned.

(30) Foreign Application Priority Data
Mar. 12, 2010 (EP) 10156372.4

(51) Int. Cl. A61K 9/107; A61K 31/436; A61K 47/34

(52) U.S. Cl. CPC A61K 9/1075; A61K 47/34; A61K 31/436

(57) ABSTRACT

The present invention relates to a micelle composition comprising a hydrophobic compound and an amphiphilic block copolymer, wherein the amphiphilic block copolymer consists of a hydrophobic block A and a hydrophilic block B, the hydrophobic block A comprises at least one hydrophobic polymeric unit X and the hydrophilic block B comprises at least one hydrophilic polymeric unit Y whereby the X and Y blocks alternate.

The present invention further relates to a process for the preparation of the micelle composition wherein the process comprises the steps of:
a) dissolving the hydrophobic compound and the amphiphilic block copolymer in an organic solvent to form a solution,
b) adding said organic solution into an aqueous medium,
c) optionally repeating aforementioned steps.

The micelle composition according to the present invention is useful in medical applications such as therapeutic cardiovascular applications, veterinary applications, food processing applications, flame retardant applications, coatings, adhesives and cosmetics, fabric/textiles, industrial and art applications.

This application is a continuation of commonly owned co-pending U.S. application Ser. No. 13/634,445, filed Nov. 26, 2012 (now abandoned), which is the national phase application under 37 USC §371 of PCT/EP2011/053817, filed Mar. 14, 2011 which designated the U.S. and claims priority to EP 10156372.4, filed Mar. 12, 2010, the entire contents of each of which are hereby incorporated by reference.

The present invention relates to micelle compositions based on amphiphilic block copolymers. The present invention also relates to a process for the preparation of the micelle compositions suitable for medical and/or veterinary use. The invention also relates to articles or devices comprising the micelle composition.

The field of the present invention is the area of formulating hydrophobic compounds for use in aqueous systems, in particular, the formulation of relatively insoluble and/or toxic hydrophobic compounds such as cardiovascular drugs, anticancer agents, flavoring agents, vitamins, imaging agents, pigments, flame retardants, agricultural chemicals, fungicides, pesticides or insecticides.

Currently, potentially hydrophobic compounds have properties that can result in their classification as “challenging” (poorly-water-soluble) compounds. Such molecules have favorable in vitro capabilities, however due to characteristics such as poor aqueous solubility, toxicity, chemical instability, and limited cellular permeability, these compounds require formulation to be effective (Davis, S. S. et al. (1998) Int. J. Pharm. 179, 2).

Micelle systems based on amphiphilic block copolymers have been used to formulate such challenging compounds (Jones, M. C et al. (1999) Eur. J. Pharm. Biopharm. 48, 101). The amphiphilic block copolymers comprised of hydrophobic and hydrophilic blocks, can assemble into a microphase separated, core/shell architecture in a selective solvent. In an aqueous environment, the hydrophobic compound will be encapsulated into the hydrophobic core of the micelle while the aqueous solubility is provided by the shell of the micelle. Due to their nanoscopic dimensions and properties imparted by the shell, micelles may have long-term circulation capabilities. WO-A-9710849 discloses biodegradable polymeric micelle-type drug compositions and method for the preparation of micelles comprising water insoluble drugs which micelles are composed of amphiphilic di- or tri-block copolymers containing poly(ethylene oxide) as hydrophilic block and poly(-ξ-caprolactone) as hydrophobic block. The molecular weight of the amphiphilic block copolymer used to form the micelles is in the range of about 1430 to 6000 Daltons. The resulting micelle-drug composition may be suitable for the sustained release of the water-insoluble drugs in vivo and this effect can be maximized by controlling the molecular weights and the relative ratio of the hydrophilic and hydrophobic blocks. WO-A-9710849 discloses different PLLA-PEO block copolymers and their water solubility's. The water solubility varies from 0.2 g/100 ml to over 20 g/100 ml. A disadvantage of these micelles, which are water soluble, is their tendency to aggregate so that the stability of the micelles on the longer term can not be assured.

WO-A-05118672 discloses micelles for the administration of hydrophobic drugs formed from self-assembly of poly(ethylene oxide)-b-poly(ξ-caprolactone) (PEO-b-PCL) block copolymers with a molecular weight above 6000 Dalton. It was found that the use of higher molecular weight block copolymers in the preparation of the micelles results in less aggregation of micelle particles and a modified biodistribution. This application is however silent about the stability and water solubility of the micelles.

There is a long felt need in the art for compositions for encapsulating poorly (water) soluble compounds for use in pharmaceutical, food, cosmetic and industrial formulations. Desirably the encapsulated materials are nanoscopic in size, thermodynamically and kinetically stable, protect the hydrophobic compounds from self-aggregation and provide advantageous release rates.

Therefore the object of the present invention is to provide a micelle composition comprising amphiphilic block copolymers which result in nanoscopic micelles which are thermodynamically and kinetically stable and which protect the hydrophobic compounds from self-aggregation and provide advantageous release properties.

The object of the present invention is achieved by providing a micelle composition comprising an amphiphilic block copolymer containing a hydrophobic block A and a hydrophilic block B composed of monomeric units, whereby the ratio R of the number average molecular weight (Mₙ) of block A (Mₙ A) divided to the number average molecular weight of block B (Mₙ B) is higher than 0.95 and whereby the amphiphilic block copolymer is characterised by a parameter α whereby
3<α<5.5;
α=M_{n A}/(M_{nA}+M_{nB})×K_{o/w A}×√Mₜₒₜ in which
M_{nA}=number average molecular weight (Mₙ) of block A
M_{nB}=number average molecular weight (Mₙ) of block B
K_{o/w A}=Octanol/water partition coefficient of the monomeric units of hydrophobic block A
Mₜₒₜ=M_{nA}+M_{nB}

Unexpectedly it has been found that micelles can be prepared with an optimum in the amount and the number average molecular weight of the hydrophilic and hydrophobic blocks A and B. It has surprisingly been found that stable micelles can be provided even on the longer term, whereby the tendency of the micelles to aggregate has been reduced markedly. Due to the stability of the micelle compositions, the micelles will exhibit enhanced properties on controlled release, shelf-life and exhibiting long circulation times in vivo. Moreover and at the same time the concentration of a drug in the micelle composition can be tailored to meet dosage needs. The micelle compositions of the present invention are capable of controlling the drug release. Such micelle compositions can offer several advantages over conventional dosage forms such as, decreased systemic side effects, and extended effective residence time of the drug, enhanced efficacy (targeted release) and patient's compliance, maintenance of therapeutic levels of the drug for longer time and with narrower fluctuations of drug's concentration in the plasma.

The amphiphilic blockcopolymer preferably comprise at least a hydrophobic block A and a hydrophilic block B, the hydrophobic block A comprises at least one hydrophobic polymer X and the hydrophilic block B comprises at least one hydrophilic polymer Y whereby the X and Y units alternate. The hydrophobic polymer X and the hydrophilic polymer Y are composed of monomers.

If α<3, the amphiphilic blockcopolymers become water soluble, which leads to aggregation and instable micelles. If α>5.5 the micelles become unstable. The octanol/water partition coefficient in parameter α is the ratio of the concentrations of a monomer in the two phases of a mixture of two immiscible solvents at equilibrium. Hence these coefficients are a measure of differential solubility of the monomer between these two solvents. Appropriate alternatives for the phrase “Partition Coefficient” are “partition constant”, “partition ratio” or “distribution ratio”. Normally one of the solvents chosen is water while the second solvent is hydrophobic for example octanol. Hence the partition coefficient is a measure of how “water loving” or “water fearing” a chemical substance is. The octanol-water partition coefficient can be expressed as Log P of a solute which is to be determined using the shake-flask method at a temperature of 25° C. and a pressure of 1 bar. It consists of dissolving some of the solute, in the present invention the monomer Z of which hydrophobic polymer X is composed, in a volume of octanol and water, shaking the mixture and then measuring the concentration of the monomer Z in each solvent. The concentration of the monomer Z can be measured using UV/VIS spectroscopy. Log P=log [Z]_{octanol}/[Z]_{water}

This means that [Z] in the present invention is the concentration of the monomer, from which hydrophobic polymer X is composed, in octanol or water. If X is polylactide the monomer Z is lactic acid and the K_{o/w} of lactic acid is 1 at 25° C. and a pressure of 1 bar. If X is polycaprolacton the monomer Z is caprolacton and the K_{o/w} of caprolacton is 3 at 25° C. and a pressure of 1 bar. If X is polylactic-glycolic acid the monomer Z is lactic acid+glycolic acid and the K_{o/w} of lactic acid+glycolic acid is 1.6 at 25° C. and a pressure of 1 bar. Further examples of the hydrophobic polymers X are given below.

The hydrophobic polymer X and the hydrophilic polymer Y are preferably chosen such that the resulting amphiphilic block copolymer has a solubility in water S_{w} of less than 0.1 g/100 ml, more preferably less than 0.01 g/100 ml, most preferably 0.001 g/100 ml. The lower the solubility of the amphiphilic block copolymer in water, the more stable micelles can be prepared. Even most preferred the amphiphilic block copolymer is water insoluble. Water solubility of amphiphilic blockcopolymers can be measured as for example disclosed in WO9710849, which is incorporated by reference.

The number average molecular weight of the hydrophobic polymer X and hydrophilic polymer Y can be measured via Gel permeation chromatography NMR. End group analysis by NMR offers an easy method for molecular weight (avg. chain length) determination of polymers using an instrument commonly found in many analytical labs and it can also be used to determine the molecular weight of block-copolymer molecules. Sensitivity of the instrument and the subsequent ability to detect end-group protons and the monomer unit protons between the two blocks will determine the upper limit that can be measured. The method relies on a few simple needs such as identifiable end-group and “inter blocks” protons distinguishable from repeating monomer group protons by NMR, accurate integration of these protons and knowledge of monomer formula weights. Once the ratio of protons on the end-groups to protons on the polymer chain is determined, the Mₙ value can be generated. For the outer blocks in the tri-block copolymer this would be the number of the repeating units multiplied by the molecular weight of the repeating unit+the molecular weight of the end-groups. The number of repeating units is determined from the ratio of the integral of the repeating unit protons and the integral of the end-group protons where both are normalized to an integral per proton. For the inner block of the tri-block a similar calculation applies but in this case not the end-group protons but the monomer unit protons between the two blocks are taken. Obviously, also a different molecular weight of the repeating unit applies. Extension to penta-block polymers involves the integration of yet an additional set of monomer unit protons between the extra blocks.

The amphiphilic blockcopolymers are for example AB di-blocks, ABA- or BAB-tri-blockcopolymers but also multi-block copolymers having repeating BA or AB blocks to make A(BA)n or B(AB)n copolymers where n is an integer of from 2 to 5 are part of the present invention. Both ABA and BAB type triblock copolymers may be synthesized by ring opening polymerization, or condensation polymerization according to reaction schemes disclosed in U.S. Pat. No. 5,683,723 and U.S. Pat. No. 5,702,717, hereby fully incorporated by reference. For example they may be prepared via ring opening polymerization of one of the cyclic ester monomers, such as lactide, glycolide, or 1,4-dioxan-2-one with monomethoxy poly(ethylene glycol) (mPEG) or poly(ethylene glycol) (PEG) in the presence of stannous octoate as a catalyst at 80˜130 Degrees C. The block copolymer product is dissolved in dichloromethane or acetone, precipitated in diethyl ether, hexane, pentane, or heptane, followed by drying.

The A blocks are composed of at least a hydrophobic polymer X which may be chosen from the group consisting of polylactides, polycaprolactone, copolymers of lactide and glycolide, copolymers of lactide and caprolactone, copolymers of lactide and 1,4-dioxan-2-one, polyorthoesters, polyanhydrides, polyphosphazines, poly(hydroxybutyrate), poly(tetramethylene carbonate) or hydrophobic poly(ester amides), poly(amino acid)s or polycarbonates. Polymer X is utilized because of its biodegradable, biocompatible, and solubilization properties. The in vitro and in vivo degradation of the hydrophobic, biodegradable polymer X is well understood and the degradation products are naturally occurring compounds that are readily metabolized and/or eliminated by the patient's body. Preferably, hydrophobic polymer X is chosen from the group consisting of polylactide, polycaprolactone, a copolymer of lactide and glycolide, a copolymer of lactide and caprolactone, and a copolymer of lactide and 1,4-dioxan-2-one. As evident in case that the hydrophobic polymer unit X is for example polylactide the monomer is lactic acid. The A block may of course also comprise more than one hydrophobic polymer X.

The number average molecular weight of the hydrophobic polymer X is preferably within the range of 500˜20,000 Daltons, and more preferably within the range of 1,000˜10,000 Daltons.

The B blocks comprise at least a hydrophilic polymer Y which may be chosen from hydrophilic polyesteramide, polyvinylalcohol or polyethylene glycol (PEG). PEG is preferably chosen as the hydrophilic, water-soluble block because of its unique biocompatibility, nontoxicity, hydrophilicity, solubilization properties. Also PEG copolymers based on the L-amino acids can be used. Examples include, without limitation, poly(ethyleneglycol)-b-poly(beta-benzyl-L-glutamate), poly(ethylene glycol)-b-poly(L-lysine acid), polyethylene glycol)-b-poly(aspartic acid, poly(ethylene glycol)-b-poly(beta-benzyl-L-aspartate), and acyl esters of the foregoing block copolymers. The number average molecular weight of the polyalkylene glycol or its derivatives is preferably within the range of 200˜20,000 Daltons and more preferably within the range of 1,000˜15,000 Daltons. The content of the hydrophilic component is within the range of 40˜80 wt percent, preferably 40˜70 wt percent, based on the total weight of the block copolymer.

Most preferably the amphiphilic block copolymer it is a triblock copolymer composed of X-Y-X. The triblock copolymer preferably comprises as polymer X polylactic acid, a hydrophobic polyesteramide or polycaprolactone and as polymerY preferably polyethyleneglycol, polyvinylalcohol or a hydrophilic polyesteramide. Specific examples include, but are not limited to PLGA-PEG-PLGA, PCL-PEG-PCL or poly(L-amino acid)-PEG-poly(L-amino acid) polymers.

It was moreover found that it is possible to produce monomodal micelles compositions. This is however dependent on the water solubility S_{w} of the amphiphilic blockcopolymer. It has been found that monomodal micelle compositions can be prepared if the amphiphilic block copolymer has a very low water solubility S_{w} preferably an S_{w} of less than 0.1 g/100 ml, more preferably an S_{w} of less than 0.01 g/100 ml, most preferably an S_{w} of less than 0.001 g/100 ml.

In the context of the present invention the term of “monomodal micelle composition” refers to an unfiltered micelle composition.

In a preferred embodiment, the invention relates to monomodal micelle compositions comprising a hydrophobic compound and an amphiphilic block copolymer, wherein the amphiphilic block copolymer consists of a hydrophobic blocks A and hydrophilic blocks B whereby the block A consists of one and the same hydrophobic polymer X and hydrophilic block B consists of one hydrophilic polymer Y, whereby the X and Y blocks alternate as X-Y-X. The ratio R of the number average molecular weight (Mₙ) of block A (M_{n A}) divided to the number average molecular weight of block B (M_{n B}), is higher than 0.95. Preferably the R is higher than 1.3 more preferably higher than 1.7, even more preferably higher than 2, most preferably higher than 3, for example higher than 3.5.

The number average molecular weight of the amphiphilic block copolymer is chosen, at least in part, according to the size and flexibility of the hydrophobic compound.

The hydrophobic compound as used herein is a compound which is not freely soluble in water and which is encapsulated within the amphiphilic block copolymer according to the present invention. Examples of the hydrophobic compounds include hydrophobic drugs such as anticancer agents, antiinflammatory agents, antifungal agents, antiemetics, antihypertensive agents, sex hormones, and steroids. Typical examples of the hydrophobic drugs are: anticancer agents such as paclitaxel, camptothecin, doxorubicin, daunomycin, cisplatin, 5-fluorouracil, mitomycin, methotrexate, and etoposide; antiinflammatory agents such as indomethacin, ibuprofen, ketoprofen, flubiprofen, diclofenac, piroxicam, tenoxicam, naproxen, aspirin, and acetaminophen; antifungal agents such as itraconazole, ketoconazole, and amphotericin; sex hormons such as testosterone, estrogen, progestone, and estradiol; steroids such as dexamethasone, prednisolone, and triamcinolone; antihypertensive agents such as captopril, ramipril, terazosin, minoxidil, and parazosin; antiemetics such as ondansetron and granisetron; antibiotics such as penicillin's for example B-lactams, chloramphenicol, metronidazole and fusidic acid; cyclosporine and biphenyl dimethyl dicarboxylic acid. Other examples of hydrophobic compounds are food ingredients, vitamins, pigments, dyes, insect repellents, UV light absorbing compounds, catalysts, photo-/UV-stabilizers, fungicides, insecticides or flame retardants. In particular, the hydrophobic compound may be selected from the group of nutrients, drugs, pharmaceuticals, proteins and peptides, vaccines, genetic materials, (such as polynucleotides, oligonucleotides, plasmids, DNA and RNA), diagnostic agents, and imaging agents.

The hydrophobic compound may be capable of stimulating or suppressing a biological response. The hydrophobic compound may for example be chosen from growth factors (VEGF, FGF, MCP-1, PIGF, anti-inflammatory compounds, antithrombogenic compounds, anti-claudication drugs, anti-arrhythmic drugs, anti-atherosclerotic drugs, antihistamines, cancer drugs, vascular drugs, ophthalmic drugs, amino acids, vitamins, hormones, neurotransmitters, neurohormones, enzymes, signaling molecules and psychoactive medicaments.

More examples of hydrophobic drugs are neurological drugs (amphetamine, methylphenidate), alpha1 adrenoceptor antagonist (prazosin, terazosin, doxazosin, ketenserin, urapidil), alpha2 blockers (arginine, nitroglycerin), hypotensive (clonidine, methyldopa, moxonidine, hydralazine minoxidil), bradykinin, angiotensin receptor blockers (benazepril, captopril, cilazepril, enalapril, fosinopril, lisinopril, perindopril, quinapril, ramipril, trandolapril, zofenopril), angiotensin-1 blockers (candesartan, eprosartan, irbesartan, losartan, telmisartan, valsartan), endopeptidase (omapatrilate), beta2 agonists (acebutolol, atenolol, bisoprolol, celiprolol, esmodol, metoprolol, nebivolol, betaxolol), beta2 blockers (carvedilol, labetalol, oxprenolol, pindolol, propanolol) diuretic actives (chlortalidon, chlorothiazide, epitizide, hydrochlorthiazide, indapamide, amiloride, triamterene), calcium channel blockers (amlodipin, barnidipin, diltiazem, felodipin, isradipin, lacidipin, lercanidipin, nicardipin, nifedipin, nimodipin, nitrendipin, verapamil), anti arthymic active (amiodarone, solatol, diclofenac, enalapril, flecainide) or ciprofloxacin, latanoprost, flucloxacillin, rapamycin and analogues and limus derivatives, paclitaxel, taxol, cyclosporine, heparin, corticosteroids (triamcinolone acetonide, dexamethasone, fluocinolone acetonide), anti-angiogenic (iRNA, VEGF antagonists: bevacizumab, ranibizumab, pegaptanib), growth factor, zinc finger transcription factor, triclosan, insulin, salbutamol, oestrogen, norcantharidin, microlidil analogues, prostaglandins, statins, chondroitinase, diketopiperazines, macrocycli compounds, neuregulins, osteopontin, alkaloids, immuno suppressants, antibodies, avidin, biotin, clonazepam.

The hydrophobic drugs can be delivered for local delivery or as pre or post surgical therapies for the management of pain, osteomyelitis, osteosarcoma, joint infection, macular degeneration, diabetic eye, diabetes mellitus, psoriasis, ulcers, atherosclerosis, claudication, thrombosis viral infection, cancer or in the treatment of hernia.

In the context of the present invention the term “micelle(s)” refers only to the amphiphilic block copolymers assembled into a microphase separated, core/shell architecture in a selective organic solvent. A micelle (plural micelles, micella, or micellae) is an aggregate of amphiphilic molecules dispersed in a liquid. A typical micelle in aqueous solution forms an aggregate with the hydrophilic “head” regions in contact with surrounding solvent, sequestering the hydrophobic regions in the micelle centre. Micelles are approximately spherical in shape. Other phases, including shapes such as ellipsoids, cylinders, and rods are also possible. The shape and size of a micelle is a function of the molecular geometry of its molecules and solution conditions such as concentration, temperature, pH, and ionic strength.

The micelle composition according to the present invention may comprise a further hydrophobic core excipient such as a fatty acid, a vitamine or any hydrophobic polymer such as for example polycaprolactone. In this way the release properties can be further steered. Also the size of the micelles can be adjusted in this way.

The micelle composition of the present invention may optionally comprise a lyoprotectant. A lyoprotectant acts as a stabilizer for the loaded micelles during for example freeze drying. In this way the micelles do not coalesce so that the dried product does not readily disperse when an aqueous dispersant is added. The lyoprotectant can be a saccharide or polyol, for example, trehalose, sucrose or raffinose, or another hydrophilic polyol such as maltodextrin, fructose, glycerol, sorbitol, inositol and mannose. Lyoprotectants can also be materials other than sugars such as PEG.

Typically, the ratio of amphiphilic block copolymer to hydrophobic core excipient or lyoprotectant ranges from 1:1 w/w to about 1:50 w/w, preferably from 1:1 w/w to 1:10 w/w, advantageously to 1:5 w/w.

The lyoprotectant can be added to the solvent along with the hydrophobic compound and the amphiphilic block copolymer or it can be added to water upon bringing into water, the solution of the hydrophobic compound and the amphiphilic block copolymer formed in the organic solvent.

The micelle composition according to the present invention may be a mixture of amphiphilic blockcopolymers. The micelle composition may further comprise an amphiphilic di block copolymer containing a hydrophobic block A and a hydrophilic block B wherein the hydrophobic block A comprises at least one hydrophobic polymer X and the hydrophilic block B comprises at least one hydrophilic polymer Y. The amount of diblock copolymer may vary up to 30 wt % of the total composition.

The micelles according to the present invention comprise an average particle size in the range of 10-800 nm, preferably 15-600 nm, more preferably 20-400 nm, most preferably in the range of 25-200 nm. The desired size is strongly dependent on the application and can be adjusted accordingly. The size of the micelles was determined by Dynamic Light Scattering (DLS) (Zetasizer Nano ZS, Malvern Instruments Ltd., Malvern, UK) at 25° C. at a scattering angle of 173°.

In general, micelles can be fabricated using a variety of techniques such as spray drying, freeze spray evaporation or emulsification (co-solvent evaporation). It is known to the person skilled in the art that the physical and chemical properties of micelles fabricated via emulsification, are greatly depended on the emulsification processing steps one applies for preparing the micelles. For example WO-A-03082303 discloses a process for the preparation of micelles which micelles comprise an amphiphilic block copolymers and a hydrophobic compound, and optionally a lyoprotectant or micelles' stabilizer. The process steps for producing the micelles include dissolving the hydrophobic compound and the amphiphilic block copolymer in a volatile organic solvent and then adding water to the miscible solution, with mixing, to promote the formation of micelles and the partitioning of the hydrophobic compound into the micelle cores. The water is added slowly to induce micellization through the critical water content of the amphiphilic block copolymers (level of water required for assembly of the amphiphilic block copolymers). The water content is greater than the critical weight concentration (CWC). Subsequently, the organic solvent is removed by evaporation under reduced pressure or elevated temperature. After loading, the micelles based on the amphiphilic block copolymers can be freeze dried for later reconstitution.

One of the main disadvantages is of this process is the sensitivity to the amount and the addition rate of water up to the critical weight concentration (CWC), both being critical for the micelle formation and stability of the micelles but also for the end average particle size and particle size distribution.

Therefore it is a further object of the present invention to provide a process for the preparation of the micelles not having the above disadvantages.

The present invention further relates to a process for preparing the micelle composition wherein the process comprises the steps of:
a) dissolving the hydrophobic compound and the amphiphilic block copolymer in an organic solvent to form a solution,
b) adding said organic solution into an aqueous medium,
c) optionally repeating aforementioned steps.

In a preferred embodiment, the hydrophobic compound is a therapeutic agent.

The micelle composition may also comprise more than one hydrophobic compound.

The concentration of the amphiphilic block copolymer in the organic solvent depends on the organic solvent used. For example in case that acetone is used as a solvent the concentration of the amphiphilic block copolymer at most 130 mg/mL (milligram per litre), preferably is at most 100 mg/L, more preferably is at most 65 mg/L.

As used in the context of the present invention, an organic solvent is a water miscible liquid used to produce a solution with at least one amphiphilic block copolymer and at least one hydrophobic compound. For use in the present methods, the solvent is one which desirably has a boiling temperature lower than that of water (less than 100 degrees centigrade at 1 atm). Preferably, the organic solvent forms an azeotrope with water, advantageously a negative azeotrope. Where the solvent and water form an azeotrope, the azeoptropic mixture can be dried by removing the azeotrope under conditions of decreased pressure and/or elevated temperature. Examples include without limitation, acetone, methanol, ethanol, acetonitrile, tetrahydrofurane, propanol, isopropanol, ethyl acetate, etc.

In a preferred embodiment, the organic solvent is selected from the group consisting of acetone, tetrahydrofurane, methanol, ethanol, acetonitirile or mixtures thereof.

The aqueous medium is selected from the group consisting of water, saline solution or a buffer solution with a pH in the range of 1-14.

The process of the present invention offers enhanced control over the micelles' average particle size and distribution, the possibility to skip laborious and/or expensive process steps such as solvent evaporation, drying, sterilization, etc., Moreover the process is insensitive to the amount and/or the addition rate of water, it does not comprise a micelle's stabilizer such as a surfactant, it can be executed continuous on either small or large scale thus providing a robust, scalable and economically attractive method for the preparation of the micelle compositions.

The process of the present invention can also provide micelle compositions that can also exhibit one or more enhanced properties such as enhanced controlled release, enhanced self-life, being directly injectable and at the same time the concentration of the drug in the micelle composition can be tailored to meet dosage needs. Of course the process can be reversed to encapsulate hydrophilic compounds.

It is also possible to functionalize at least the surface of the micelles by providing at least the surface with a functional group, in particular with a signaling molecule, an enzyme or a receptor molecule, such as an antibody. The receptor molecule may for instance be a receptor molecule for a component of interest, which is to be purified or detected, e.g. as part of a diagnostic test, making use of the particles of the present invention. Suitable functionalisation methods may be based on a method known in the art.

In the context of the present invention the terms “method for the preparation” and “process” will be used interchangeably.

Unless the context clearly indicates otherwise, as used herein plural forms of the terms herein are to be construed as including the singular form and vice versa.

For all upper and lower boundaries of any parameters given herein, the boundary value is included in each range for each parameter. All combinations of minimum and maximum values of the parameters described herein may be used to define the parameter ranges for various embodiments and preferences of the invention.

It will be understood that the total sum of any quantities expressed herein as percentages cannot (allowing for rounding errors) exceed 100%. For example the sum of all components of which the composition of the invention (or part(s) thereof) comprises may, when expressed as a weight (or other) percentage of the composition (or the same part(s) thereof), total 100% allowing for rounding errors. However where a list of components is non exhaustive the sum of the percentage for each of such components may be less than 100% to allow a certain percentage for additional amount(s) of any additional compound(s) that may not be explicitly described herein.

The micelle composition of the present invention can be administered, for example oral, parenteral, buccal, sublingual, nasal, rectal, patch, pump or transdermal administration and in pharmaceutical compositions formulated accordingly. Parenteral administration includes intravenous, infraperitoneal, subcutaneous, intramuscular, transepithelial, nasal, intrapulmonary, intrathecal, rectal and topical modes of administration. Parenteral administration may be by continuous infusion over a selected period of time. The micelles of the invention can be administered orally for example, with an inert diluent or with an assimilable edible carrier, it may be enclosed in hard or soft shell gelatin capsule, it may be compressed into tablets or it may be incorporated directly with the food of the diet. For oral therapeutic administration, the micelles of the present invention may be incorporated within an excipient and used in the form of ingestible tablets, buccal tablets, troches, capsules, elixirs, suspensions, syrups, wafers, and the like. The micelle composition of the invention may also be administered parenterally. Solutions of the micelle composition according to the present invention can be prepared in water. Under ordinary conditions of storage and use, these preparations contain a preservative to prevent the growth of microorganisms. A person skilled in the art would know how to prepare suitable formulations.

The fields wherein the micelles according to the present invention can be used include dermatology, vascular, orthopedics, ophthalmic, spinal, intestinal, pulmonary, nasal, or auricular. Besides in a pharmaceutical application, the micelles according to the present invention may inter alia be used in an agricultural or food application. In particular, such micelles may comprise food additives, pesticides, insecticides or plant-nutrients.

The present invention further relates to articles comprising the micelle composition of the present invention. In another aspect, the invention provides for a device comprising the micelle composition of the present invention. In the context of the present invention, an article is an individual object or item or element of a class designed to serve a purpose or perform a special function and can stand alone.

In yet another preferred embodiment, the invention provides for a device comprising the article of the present invention. A device is a piece of equipment or a mechanism designed to serve a special purpose or perform a special function and can consist of more than one article (multi-article assembly).

Examples of devices include, but are not limited to catheters, stents, rods, implants.

In another aspect the invention provides for the use of the micelle composition of the invention, the article of the invention, the device of the invention in medical applications such as therapeutic cardiovascular applications, veterinary applications, food processing applications, flame retardant applications, coatings, adhesives and cosmetics, fabric/textiles, industrial and art applications.

In another preferred embodiment, the invention provides for a micelle composition of the present invention for use as a medicament.

In yet another preferred embodiment, the invention provides for the use of a micelle composition of the present invention for the manufacture of a medicament for cardiovascular therapeutic applications.

In another preferred embodiment the invention provides for a method for manufacturing a medicament intended for cardiovascular therapeutic applications characterized in that the micelle composition of the present invention is used.

The present invention will now be described in detail with reference to the following non limiting examples which are by way of illustration only.

### EXAMPLES

### Materials and Methods.

PLGA 20 kDa was purchased from Ingelheim Boehringer.
PCL 80 kDa was purchased from Solvay
PEG (3.0 kDa and 6.0 kDa), dexamethasone and Sn₂Oct were purchased from Sigma Aldrich.
Acetone was purchased from BASF.
Rapamycin and paclitaxol were purchased from Oscar Tropitz.
Saline was purchased from BBraun.
Intensity-based Z-average as a particle size value measured by DLS
Polydispersity (Pdl) is a measure of the width of the size distribution which is measured by the Malvern Zetasizer NanoZS.
All other solvents are of analytical grade and purchased from Merck.
Mₙ can be measured as followed. An example is given for PLGA.

Hydrolisation of PLGA with NaOH; PEG is unaffected. The hydrolysis was performed in a closed bottle (or an autoclave (Roth, Karlsruhe, Germany) for 72 h at 140° C. and 5 bar) with 2 mL PLGA or 20 mg solid sample and 200 μL 10 M NaOH solution for several days (3-7) at 90° C.

The concentration of glycolic acid and lactic acid was determined on an Agilent 1100 LC-MS system, which consists of a binary pump, degasser, autosampler, column oven, diode-array detector and a time-of-flight-MS. The ESI-MS was run in negative mode, with the following conditions: m/z 50-3200, 215 V fragmentor, 0.94 cycl/sec, 350° C. drying gas temperature, 12 L N2/min drying gas, 45 psig nebuliser pressure and 4 kV capillary voltage. UV detection was performed at 195 nm. The separation was performed with a 250×4.6 mm Prevail-C18 column (Alltech, USA) at room temperature and with a gradient of 50 mM sulfonic acid in ultra-pure water (mobile phase A) and acetonitrile (mobile phase B). The gradient was started at t=0 min with 99% (v/v) A, was stationary for 5 min and then changed linearly over 10 min to 90% (v/v) B (t=15 min). The flow rate was 0.5 mL/min and injection volume was 5 μL.

The weight-average molecular weight (Mₙ) and concentration of PEG was determined by SEC using a highly polar hydroxylated methacrylate 8×300 mm Suprema 1000 A column (10 μm particle size), with a separation range of 1-100 kDa (PSS, Mainz, Germany). The mobile phase (0.1 M NH4Ac) was pumped at a flow rate of 1.0 mL/min. The SEC analysis was performed using an Agilent 1100 LC-DAD system. Concentration and Mₙ can be analyzed using PEG calibration standards.

### Example 1

### Preparation of PLGA-PEG-PLGA Triblock Copolymers Via Ring Opening Polymerization

PEG was weighed into a two-necked round bottle flask after drying for 24 hours in a vacuum oven at 90° C. and subsequently placed in an oil bath at 150° C. A vacuum was employed for at least 60 minutes before continuing synthesis. The addition of lactide and glycolide (molar ratio of lactyl:glycolyl=50:50) was carried out by removing the vacuum and at the same time flushing with nitrogen gas. Under stirring a homogenous melt was obtained after which stannous octoate (Sn₂Oct), was added in the same way as the monomers. The reaction conditions were maintained for 20 hours where after the vacuum was replaced by nitrogen gas. The copolymers obtained in this way are listed below.

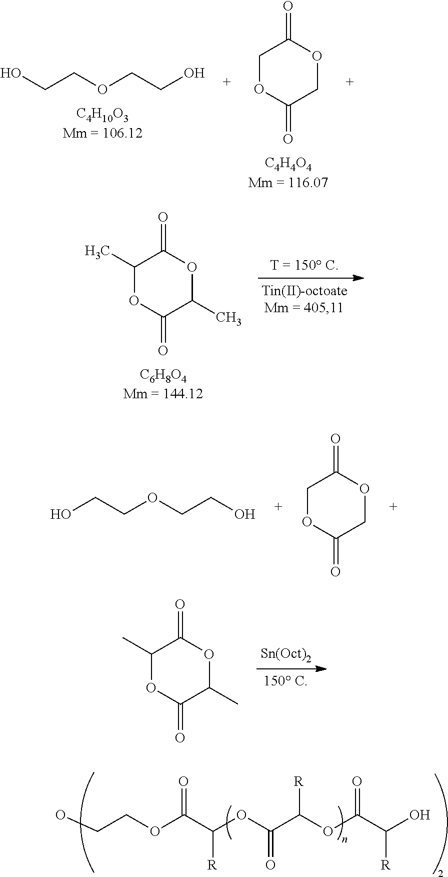

PEG-3000-diol
or
PEG-6000-diol

### Batch 1: Synthesis of PLGA(50/50)-PEG-PLGA(50/50) 7.5 k-6 k-7.5 k

| | Actual | Theory |
|---|---|---|
| D,L-Lactide | 3.9131 g | 3.96 g |
| Glycolide | 3.2471 g | 3.19 g |
| PEG-6000-diol | 2.8467 g | 2.86 g |
| Sn₂Oct | 2 drops | 4.4 mg |

### Batch 2: Synthesis of PLGA(50/50)-PEG-PLGA(50/50) 7.5 k-6 k-7.5 k

| | Actual | Theory |
|---|---|---|
| D,L-Lactide | 3.8461 g | 3.96 g |
| Glycolide | 3.1664 g | 3.19 g |
| PEG-6000-diol | 2.8597 g | 2.86 g |
| Sn₂Oct | 2 drops | 4.4 mg |

In batches 1 and 2, the amphiphilic block copolymer is a PLGA-PEG-PLGA triblock copolymers wherein M_{n A}=7.5 kDa and M_{n B}=6 kDa.

The ratio R of the number average molecular weight (Mₙ) of block A (M_{n A}) divided to the number average molecular weight of block B (M_{n B}) is 2.5 and the amphiphilic block copolymer is characterised by parameter α being 5.24 and calculated as followed:
α=M_{n A}/(M_{nA}+M_{nB})×K_{o/w A}×·Mₜₒₜ in which
M_{n A}=7.5 kDa
M_{nB}=6 kDa
K_{o/w monomeric units of A}=Octanol/water partition coefficient of lactic acid/glycolic acid is1.6.

### Batch 3: Synthesis of PLGA(50/50)-PEG-PLGA(50/50) 3.75 k-3 k-3.75 k

| | Actual | Theory |
|---|---|---|
| D,L-Lactide | 3.9098 g | 3.96 g |
| Glycolide | 3.3233 g | 3.19 g |
| PEG-3000-diol | 2.8573 g | 2.86 g |
| Sn₂Oct | 2 drops | 4.4 mg |

### Batch 4: Synthesis of PLGA(50/50)-PEG-PLGA(50/50) 3.75 k-3 k-3.75 k

| | Actual | Theory |
|---|---|---|
| D,L-Lactide | 3.9790 g | 3.96 g |
| Glycolide | 3.2620 g | 3.19 g |
| PEG-3000-diol | 2.8548 g | 2.86 g |
| Sn₂Oct | 2 drops | 4.4 mg |

In batches 3 and 4, M_{n A}=3.75 kDa and M_{n B}=3 kDa.
The ratio R=2.5 and α=3.7 and calculated as follows:
α=M_{n A}/(M_{nA}+M_{nB})×K_{o/w A}×√Mₜₒₜ in which
M_{n A}=3.75 kDa M_{nB}=3 kDa K_{o/w}=Octanol/water partition coefficient of lactic acid/glycolic acid is 1.6.

### Example 2

### Preparation of PCL-PEG-PCL Triblock Copolymers Via Ring Opening Polymerization

PEG was charged with ε-caprolactone in a 100 ml round bottomed flask. The reaction mixture was heated to 100° C. and stirred till a homogenous mixture was formed. A catalyst stock solution of tin(II)octoate (58.1 mg, 0.143 mmol) was prepared in hexane (5 mL). 1 mL of the catalyst stock solution was added to the reaction mixture at 100° C. The reaction mixture was further heated to 150° C. for an additional 18 hours (overnight) to allow the reaction to proceed. The following morning the reaction mixture was cooled to room temperature, an off white waxy solid material was obtained.

### Batch 1: Synthesis of PCL-PEG-PCL 1.5 k-3.0 k-1.5 k

| | |
|---|---|
| Actual | Theory |

| | | | | |
|---|---|---|---|---|
| ε-caprolactone | 14.9980 | g | 0.131 | mol |
| PEG-3000-diol | 15.0004 | g | 5.0 | mmol |

Sn₂Oct-hexane solution (1 mL used in synthesis)

| | | | | |
|---|---|---|---|---|
| Sn₂Oct | 58.1 | mg | 0.143 | mmol |
| Hexane | | | 5 | mL |

In batch 1 the amphiphilic block copolymer is PCL-PEG-PCL triblock copolymer wherein
M_{n A}=1.5 kDa and M_{n B}=3 kDa.
The ratio R=1 and α=3.67 and calculated as follows:
α=M_{n A}/(M_{nA}+M_{nB})×K_{o/w A}×√Mₜₒₜ in which
M_{n A}=1.5 kDa
M_{nB}=3 kDa
K_{o/w monomeric units of A}=Octanol/water partition coefficient of caprolacton is 3.

### Batch 2: Synthesis of PCL-PEG-PCL 1.7 k-3.0 k-1.7 k

| | |
|---|---|
| Actual | Theory |

| | | | | |
|---|---|---|---|---|
| ε-caprolactone | 15.9433 | g | 0.137 | mol |
| PEG-3000-diol | 14.0628 | g | 4.7 | mmol |

Sn₂Oct-hexane solution (1 mL used in synthesis)

| | | | | |
|---|---|---|---|---|
| Sn₂Oct | 58.1 | mg | 0.143 | mmol |
| Hexane | | | 5 | mL |

In batch 2, M_{n A}=1.7 kDa and M_{n B}=3 kDa.
The ratio R=1.13 and α=4.03 and calculated as follows:
α=M_{n A}/(M_{nA}+M_{nB})×K_{o/w A}×√Mₜₒₜ in which
K_{o/w monomeric units of A}=Octanol/water partition coefficient of caprolacton is 3.

### Example 3

### Purification of the Synthesized Triblock Copolymers

The triblock copolymers of examples 1 and 2 were dissolved in acetone at a weight percentage of 10-20% and filtered over an Acrodisc premium 25 mm Syringe filter, G×F/0.45 μm PVDF membrane, to remove particulate impurities and dust particles, which can interfere with the nanoprecipitation process. Hereafter the filtered solution was collected into a beaker of 500 mL PTFE and evaporated to remove the solvent over night (10-12 hours) at maximum 40° C. and minimum 300 mbar.

The blockcopolymers were characterized by ¹H-NMR and GPC see

TABLE 1
Table 1: Tri-block copolymers composition

| PLGA-block | PEG-block | PLGA-block |
|---|---|---|
| 7.5 kDa | 6 kDa | 7.5 kDa |
| 3.75 kDa | 3 kDa | 3.75 kDa |
| 7.5 kDa | 3 kDa | 7.5 kDa |
| PCL-block | PEG-block | PCL-block |
| 1.5 kDa | 3 kDa | 1.5 kDa |
| 1.7 kDa | 3 kDa | 1.7 kDa |
| 1.9 kDa | 3 kDa | 1.9 kDa |
| 1.9 kDa | 4 kDa | 1.9 kDa |
| 3.8 kDa | 4 kDa | 3.8 kDa |
| 3.8 kDa | 6 kDa | 3.8 kDa |

### Example 4

### Preparation of a Drug Loaded Micelle Composition Based on PLGA-PEG-PLGA

855 mg (PLGA 3.75 k)₂-PEG 3 k was dissolved in 14.25 ml Acetone selectipur. The solution was filtered over a 0.45 μm filter to remove dust particles. 183.85 mg Rapamycin and 183.85 mg PLGA-PTE 20 k were dissolved in the (PLGA 3.75 k)₂-PEG 3 k/acetone solution. ([Rapa]=12.9 mg/ml).

The formulation was filtered over a 0.45 μm filter to remove dust particles.

1 ml of the filtered formulation was pipetted into 25 ml of MilliQ water and measured by Dynamic light scattering (DLS).

Results and properties of the micelle composition are given in table 2.

### Example 5

### Preparation of a Drug Loaded Micelle Composition Based on PCL-PEG-PCL

1439.14 mg (PCL2 k)₂-PEG 3 k was dissolved in Acetone selectipur (60 mg/ml). The polymer was filtered over a 0.45 μm filter.

Rapamycin and PCL 80 k was dissolved in the (PCL 2 k)₂-PEG 3 k/Acetone solution. The formulation was filtered over a 0.45 μm filter. 1 ml of the filtered formulation was pipetted into 25 ml of MilliQ water and measured by DLS.

Results and properties of the micelle composition are given in table 2.

TABLE 2

| Micelle | Shell | | | | | | |
|---|---|---|---|---|---|---|---|
| composition | material | Core material | | Z-average | PdI | Width | Distribution |
| Example 4 | (PLGA 3.75k)₂- | PLGA- | Rapamycine | 93.15 | 0.185 | 56.44 | Monomodal |
| | PEG 3k | PTE 20k | 50% | | | | |
| | | 50% | | | | | |
| Example 5 | (PCL 2k)₂- | PCL 80k | Rapamycine | 78.67 | 0.245 | 58.64 | Monomodal |
| | PEG 3k | 50% | 50% | | | | |

### Example 6

### Preparation of Micelle Compositions Based on PLGA-PEG-PLGA

A. Micelles Made from Different Concentrations Triblockcopolymer.
a. 32.1 mg PEG (6 k)-(PLGA (7.5 k))₂ was dissolved in 1 ml acetone, 0.400 ml of the solution was added to 10 ml Milli Q.
b. 64.5 mg PEG (6 k)-(PLGA (7.5 k))₂ was dissolved in 1 ml acetone, 0.400 ml of the solution was added to 10 ml Milli Q.
   Results and properties of the micelle composition are given in table 3.

### B. Micelles at Different pH Values

a. 64.5 mg PEG (6 k)-(PLGA (7.5 k))₂ was dissolved in 1 ml acetone, 0.400 ml of the solution was added to 10 ml pH buffer (CertiPUR buffer: citric acid/sodium hydroxide/hydrogen chloride), pH=4.
b. 64.5 mg PEG (6 k)-(PLGA (7.5 k))₂ was dissolved in 1 ml acetone, 0.400 ml of the solution was added to 10 ml pH buffer (CertiPUR buffer: boric acid/potassium chloride/sodium hydroxide), pH=9.
   Results and properties of the micelle composition are given in table 3.

### C. Micelles in Different Salt Solution

a. 64.5 mg PEG (6 k)-(PLGA (7.5 k))₂ was dissolved in 1 ml acetone, 0.400 ml of the solution was added to 10 ml 0.9% NaCl.
   Results and properties of the micelle composition are given in table 3.

### D. Micelles in Salt and pH Controlled Solution

a. 64.5 mg PEG (6 k)-(PLGA (7.5 k))₂ was dissolved in 1 ml acetone, 0.400 ml of the solution was added to 10 ml PBS, pH=7.4 (=sodium chloride/potassium chloride/sodium phosphate)
   Results and properties of the micelle composition are given in table 3.

TABLE 3
Properties of the micelle compositions

| Micelles | Aa | Ab | Ba | Bb | C | D |
|---|---|---|---|---|---|---|
| Z-average (nm) | 42.96 | 52.49 | 92.23 | 58.5 | 49.09 | 58.38 |
| PdI | 0.134 | 0.187 | 0.387 | 0.162 | 0.193 | 0.167 |
| Width (nm) | 19.7 | 32.02 | 110.1 | 24.83 | 11.71 | 29.09 |
| Distribution | Mono | Mono | Mono | Mono | Mono | Mono |
| Measuring | 25.0 | 25.0 | 25.0 | 25.0 | 25.0 | 25.0 |
| temperature | | | | | | |
| (C.) | | | | | | |

### Example 7

### Size Stability of Micelle Compositions in Time

164.1 mg (PLGA 7.5 k)₂-PEG 6 k was dissolved in 2.400 ml Acetone selectipur. The solution was filtered over a 0.45 μm filter to remove dust particles.

0.8 mg Rapamycin was dissolved in 0.800 ml acetone solution.

The formulation was filtered over a 0.45 μm filter to remove dust particles.

0.3000 ml of the (PLGA 7.5 k)₂-PEG 6 k-acetone solution was mixed with 0.100 ml of the rapamycin-acetone solution, resulting in 0.400 ml of (PLGA 7.5 k)₂-PEG 6 k/Rapamycin-acetone solution

The 0.400 ml of the of (PLGA 7.5 k)₂-PEG 6 k/Rapamycin-acetone solution was pipetted into 10 ml of MilliQ water and measured by Dynamic light scattering (DLS).

| Time (days) | z-average (nm) | Pdl |
|---|---|---|
| Day 1: | 45.28 | 0.202 |
| Day 2: | 43.81 | 0.197 |
| Day 15: | 45.27 | 0.113 |

### Example 8

### Size Stability of Micelle Compositions in Time

164.1 mg (PLGA 7.5 k)₂-PEG 6 k was dissolved in 2.400 ml Acetone selectipur. The solution was filtered over a 0.45 μm filter to remove dust particles.

0.3000 ml of the (PLGA 7.5 k)₂-PEG 6 k-acetone solution was mixed with 0.100 ml of acetone solution, resulting in 0.400 ml of (PLGA 7.5 k)₂-PEG 6 k-acetone solution

The 0.400 ml of the of (PLGA 7.5 k)₂-PEG 6 k-acetone solution was pipetted into 10 ml of MilliQ water and measured by Dynamic light scattering (DLS).

| Time (days) | z-average (nm) | Pdl |
|---|---|---|
| Day 1: | 48.69 | 0.188 |
| Day 15: | 49.08 | 0.100 |

1. A micelle composition comprising an amphiphilic triblock copolymer containing a hydrophobic block A and a hydrophilic block B, wherein
a ratio R of the number average molecular weight (Mₙ) of block A (M_{n A}) divided to the number average molecular weight of block B (M_{n B}) is higher than 0.95, and wherein
the amphiphilic block copolymer is characterised by a parameter α wherein;
3<α<5.5;
α=M_{n A}/(M_{n A}+M_{n B})×K_{o/w A}×√Mₜₒₜ in which
M_{n A}=number average molecular weight (Mₙ) of block A;
M_{n B}=number average molecular weight (Mₙ) of block B;
K_{o/w A}=octanol/water partition coefficient of the monomeric units of hydrophobic block A; and
Mₜₒₜ=M_{A}+M_{B}, wherein Mₜₒₜ is expressed in kDa, and wherein
the hydrophobic block A comprises at least one hydrophobic polymer X and the hydrophilic block B comprises at least one hydrophilic polymer Y.

2. The micelle composition according to claim 1, further comprising a hydrophobic compound.

3. The micelle composition according to claim 1, wherein the average particle size of the micelles is in the range of 10-200 nm.

4. The micelle composition according to claim 2, wherein the hydrophobic compound is selected from the group consisting of therapeutic agents, cardiovascular drugs, vitamins, flavour agents, food ingredients, pigments, catalysts, photo-or UV-stabilizers, fungicides, insecticides, flame retardants and anticancer drugs.

5. The micelle composition according to claim 4, wherein the hydrophobic compound is a cardiovascular drug.

6. The micelle composition according to claim 1, wherein the hydrophobic polymer X is selected from the group consisting of poly(lactic acid), poly(D,L-lactide-co-glycolide), poly(ε-caprolactone), poly(hydroxybutyrate), poly(tetramethylene carbonate) and poly(ester amides).

7. The micelle composition according to claim 1, wherein the hydrophilic polymer Y is selected from the group consisting of poly(ethylene oxide), poly(ester amide), polyvinylpyrrolidone and polyvinylacetate.

8. The micelle composition according to claim 1, wherein the amphiphilic triblock copolymer is a triblock copolymer comprising X-Y-X.

9. The micelle composition according to claim 8, wherein the triblock copolymer comprises polylactic acid, a hydrophobic polyesteramide or polycaprolactone as the hydrophobic polymer X, and polyethyleneglycol or a hydrophilic polyesteramide as the hydrophilic polymer Y.

10. The micelle composition according to claim 1, wherein the micelle composition further comprises a further hydrophobic core excipient.

11. The micelle composition according to claim 1, wherein the micelle composition further comprises an amphiphilic diblock copolymer containing a hydrophobic block A and a hydrophilic block B, wherein the hydrophobic block A comprises at least one hydrophobic polymeric unit X and the hydrophilic block B comprises at least one hydrophilic polymeric unit Y.

12. The micelle composition according to claim 11, wherein the diblock copolymer is present in an amount up to 30 wt % of the total composition.

13. A process for the preparation of the micelle composition according to claim 2, wherein the process comprises the steps of:
(a) dissolving the hydrophobic compound and the amphiphilic block copolymer in an organic solvent to form an organic solution,
(b) adding the organic solution into an aqueous medium, and
(c) optionally repeating steps (a) and (b).

14. The process according to claim 13, wherein the process further comprises the following steps:
(d) evaporating the organic solvent thus forming an aqueous solution,
(e) optionally repeating step (d) with the steps (a) and (b),
(f) filtering the aqueous solution to obtain the micelle composition, and
(g) optionally drying the micelles.

15. The process according to claim 13 wherein the aqueous medium is selected from the group consisting of water, saline solution and a buffer solution with a pH in the range of 1-14.

16. The process according to claim 13, wherein the organic solvent is at least one selected from the group consisting of acetone, tetrahydrofuran, methanol, ethanol, and acetonitirile.

17. An article comprising the micelle composition according to claim 1.

18. A device comprising the micelle composition according to claim 1.

19. A device comprising the article of claim 18.

20. The device according to claim 19, wherein the device is a device for medical applications, food processing applications, flame retardancy applications, coatings, adhesives and cosmetics, fabric/textiles, industrial applications and art applications.

21. The device according to claim 20, wherein the micelle composition is present in an amount that allows the micelle composition to exhibit controlled release properties.

22. A medicament which comprises the micelle composition according to claim 1.

23. The medicament according to claim 22, wherein the medicament is a cardiovascular medicament.

24. The medicament according to claim 22, wherein the medicament is a cancer treatment medicament.
